(19) United States
(12) Patent Application Publication
(10) Pub. No.: US 2016/0243352 A1
(43) Pub. Date: Aug. 25, 2016

(54) SYSTEMS AND METHODS FOR ELECTRODE ASSEMBLIES

(71) Applicant: PACESETTER, INC., Sylmar, CA (US)

(72) Inventors: Aaron Raines, Dallas, TX (US); Shichan Chiang, Valencia, CA (US); Jerome Boogaard, Forest Grove, OR (US); Serdar Unal, Los Angeles, CA (US)

(21) Appl. No.: 14/629,325

(22) Filed: Feb. 23, 2015

(51) Int. Cl. A61N 1/05; B23K 15/00; H01R 43/02; B23K 26/22

(52) U.S. Cl. CPC A61N 1/0556; B23K 26/22; B23K 15/008; H01R 43/0221; B23K 2201/36

(57) ABSTRACT

The present disclosure provides electrode assemblies. An electrode assembly includes a wire and a substantially cylindrical electrode including a radially inner surface, a radially outer surface, and a strip defined by at least one slot extending from the radially inner surface to the radially outer surface, wherein the wire is welded to the radially outer surface of the strip.

### FIELD OF THE DISCLOSURE

The present disclosure relates generally to electrode assemblies, and more particularly, to electrode assemblies for use in neurostimulation systems.

### BACKGROUND ART

Neurostimulation is a treatment method utilized for managing the disabilities associated with pain, movement disorders such as Parkinson's Disease (PD), dystonia, and essential tremor, and also a number of psychological disorders such as depression, mood, anxiety, addiction, and obsessive compulsive disorders. Deep brain stimulation systems are neurostimulation systems that deliver stimulation to a patient's brain.

Neurostimulation systems generally include leads having one or more electrodes. To control those electrodes, wires or cables are electrically coupled to the electrodes. In at least some known systems, wires or cables are electrically coupled to the electrodes using blind resistance or laser welds. However, such welds may be difficult to form, and may be relatively difficult to inspect, as the formed welds are not readily visible. In other known systems, the wire or cable may be crimped under the electrode. However, this is relatively difficult to implement due to the amount of space required for both creating and positioning the crimp.

### BRIEF SUMMARY OF THE DISCLOSURE

In one embodiment, the present disclosure is directed to an electrode assembly. The electrode assembly includes a wire and a substantially cylindrical electrode including a radially inner surface, a radially outer surface, and a strip defined by at least one slot extending from the radially inner surface to the radially outer surface, wherein the wire is welded to the radially outer surface of the strip.

In another embodiment, the present disclosure is directed to a neurostimulation system. The neurostimulation system includes an implantable pulse generator, a substantially cylindrical electrode including a radially inner surface, a radially outer surface, and a strip defined by at least one slot extending from the radially inner surface to the radially outer surface, and a wire electrically coupling the implantable pulse generator to the electrode, wherein the wire is welded to the radially outer surface of the strip.

In another embodiment, the present disclosure is directed to a method of assembling an electrode assembly. The method includes threading a wire through at least one slot defined in a substantially cylindrical electrode that includes a radially inner surface, a radially outer surface, and a strip defined by the at least one slot extending from the radially inner surface to the radially outer surface, and welding the wire to the radially outer surface of the strip.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIGS. 2A-2C are schematic views of stimulation portions that may be used with stimulation system of FIG. 1.

FIG. 3 is a perspective view of one embodiment of an electrode assembly that may be used with the stimulation system of FIG. 1.

FIG. 4 is a perspective view illustrating forming a weld in the electrode assembly of FIG. 3.

FIG. 5 is a perspective view of an alternative electrode assembly that may be used with the stimulation system of FIG. 1.

FIG. 6 is a perspective view of an alternative electrode assembly that may be used with the stimulation system of FIG. 1.

FIG. 7 is a perspective view of an alternative electrode assembly that may be used with the stimulation system of FIG. 1.

FIG. 8 is a perspective view of an alternative electrode assembly that may be used with the stimulation system of FIG. 1.

FIG. 9 is a perspective view of one embodiment of an electrode formed using a progressive die.

Corresponding reference characters indicate corresponding parts throughout the several views of the drawings.

### DETAILED DESCRIPTION OF THE DISCLOSURE

The present disclosure provides electrode assemblies. An electrode assembly includes a wire and a substantially cylindrical electrode including a radially inner surface, a radially outer surface, and a strip defined by at least one slot extending from the radially inner surface to the radially outer surface, wherein the wire is welded to the radially outer surface of the strip. Although the embodiments described herein are generally described in connection with neurostimulation systems, those of skill in the art will appreciate that the electrode assemblies described herein may be utilized in a variety of fields/applications.

Neurostimulation systems are devices that generate electrical pulses and deliver the pulses to nerve tissue of a patient to treat a variety of disorders. Spinal cord stimulation (SCS) is the most common type of neurostimulation within the broader field of neuromodulation. Deep brain stimulation (DBS) is another type of neurostimulation. In SCS, electrical pulses are delivered to nerve tissue in the spine typically for the purpose of chronic pain control. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain. SCS systems generally include a pulse generator and one or more leads. A stimulation lead includes a lead body of insulative material that encloses wire conductors. The distal end of the stimulation lead includes multiple electrodes that are electrically coupled to the wire conductors. The proximal end of the lead body includes multiple terminals (also electrically coupled to the wire conductors) that are adapted to receive electrical pulses. The distal end of a respective stimulation lead is implanted within the epidural space to deliver the electrical pulses to the appropriate nerve tissue within the spinal cord that corresponds to the dermatome(s) in which the patient experiences chronic pain. The stimulation leads are then tunneled to another location within the patient's body to be electrically connected with a pulse generator or, alternatively, to an “extension.”

The pulse generator is typically implanted within a subcutaneous pocket created during the implantation procedure. In SCS, the subcutaneous pocket is typically disposed in a lower back region, although subclavicular implantations and lower abdominal implantations are commonly employed for other types of neuromodulation therapies.

Peripheral nerve field stimulation (PNFS) is another form of neuromodulation. The basic devices employed for PNFS are similar to the devices employed for SCS including pulse generators and stimulation leads. In PNFS, the stimulation leads are placed in subcutaneous tissue (hypodermis) in the area in which the patient experiences pain. Electrical stimulation is applied to nerve fibers in the painful area. PNFS has been suggested as a therapy for a variety of conditions such as migraine, occipital neuralgia, trigeminal neuralgia, lower back pain, chronic abdominal pain, chronic pain in the extremities, and other conditions.

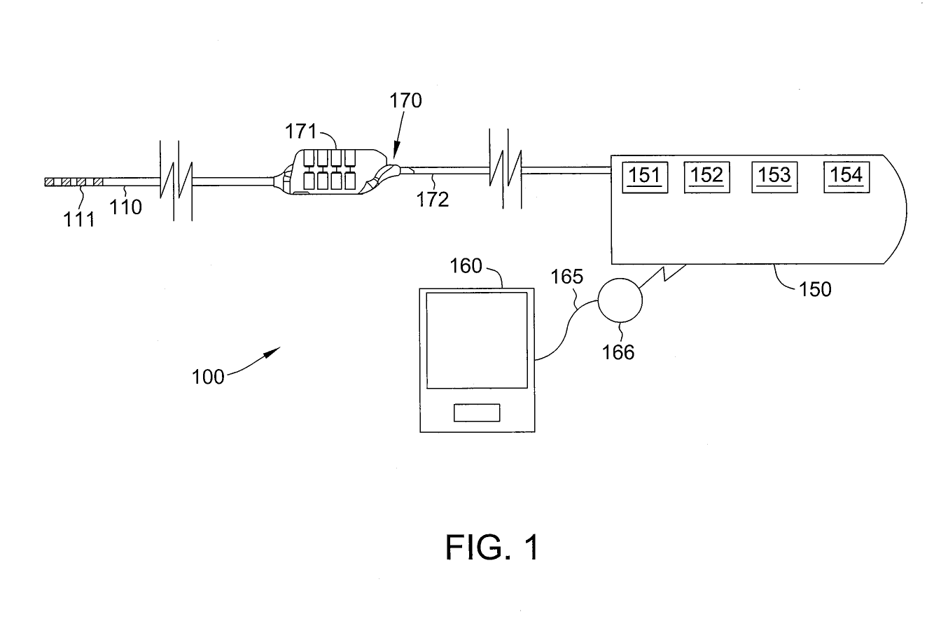

Referring now to the drawings and in particular to FIG. 1, a stimulation system is indicated generally at 100. Stimulation system 100 generates electrical pulses for application to tissue of a patient, or subject, according to one embodiment. System 100 includes an implantable pulse generator (IPG) 150 that is adapted to generate electrical pulses for application to tissue of a patient. Implantable pulse generator 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of pulse generator 150 for execution by the microcontroller or processor to control the various components of the device.

Pulse generator 150 may comprise one or more attached extension components 170 or be connected to one or more separate extension components 170. Alternatively, one or more stimulation leads 110 may be connected directly to pulse generator 150. Within pulse generator 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from pulse generator 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within pulse generator 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within pulse generator 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number of electrodes 111, terminals, and internal conductors.

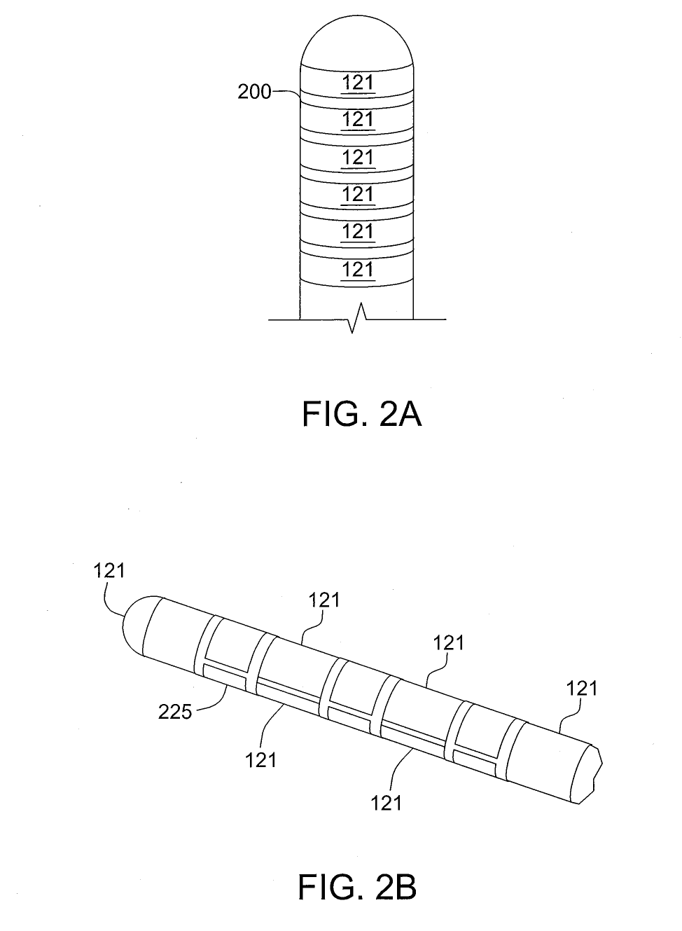

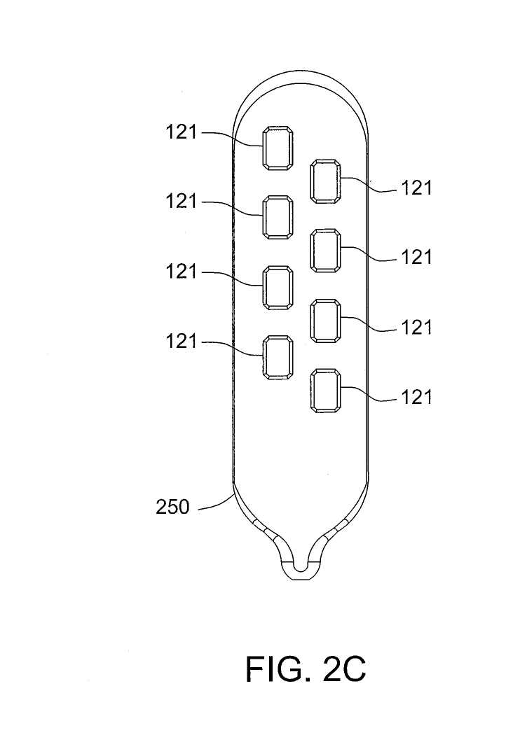

FIGS. 2A-2C respectively depict stimulation portions 200, 225, and 250 for inclusion at the distal end of lead 110. Stimulation portion 200 depicts a conventional stimulation portion of a “percutaneous” lead with multiple ring electrodes. Stimulation portion 225 depicts a stimulation portion including several “segmented electrodes.” The term “segmented electrode” is distinguishable from the term “ring electrode.” As used herein, the term “segmented electrode” refers to an electrode of a group of electrodes that are positioned at the same longitudinal location along the longitudinal axis of a lead and that are angularly positioned about the longitudinal axis so they do not overlap and are electrically isolated from one another. Example fabrication processes are disclosed in U.S. Patent Publication No. 2011/0072657, entitled, “METHOD OF FABRICATING STIMULATION LEAD FOR APPLYING ELECTRICAL STIMULATION TO TISSUE OF A PATIENT,” which is incorporated herein by reference. Stimulation portion 250 includes multiple planar electrodes on a paddle structure.

Controller device 160 may be implemented to recharge battery 153 of pulse generator 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of generator 150. The charging circuitry may also communicate status messages to controller 160 during charging operations using pulse-loading or any other suitable technique. For example, controller 160 may communicate the coupling status, charging status, charge completion status, etc.

External controller device 160 is also a device that permits the operations of pulse generator 150 to be controlled by user after pulse generator 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate pulse generator 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/93953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference.

Example commercially available neurostimulation systems include the EON MINI™ pulse generator and RAPID PROGRAMMER™ device from St. Jude Medical, Inc. (Plano, Tex.). Example commercially available stimulation leads include the QUATTRODE™, OCTRODE™, AXXESS™ LAMITRODE™, TRIPOLE™, EXCLAIM™, and PENTA™ stimulation leads from St. Jude Medical, Inc.

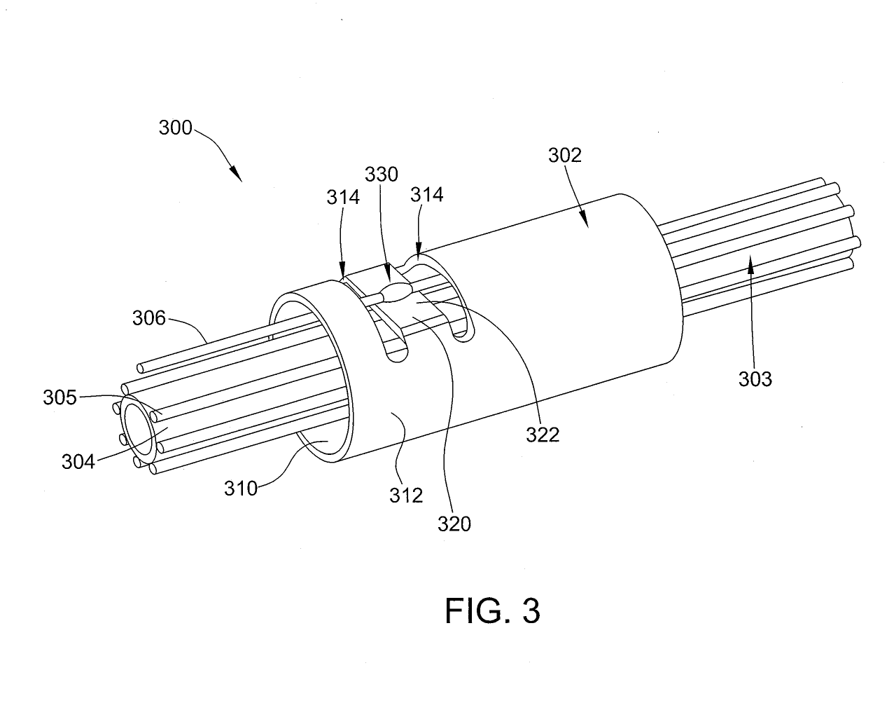

In FIG. 3, an electrode assembly is indicated generally at 300. Electrode assembly 300 may be used, for example, in stimulation portions 200, 225, and/or 250. As shown in FIG. 3, electrode assembly 300 includes a substantially cylindrical electrode 302 and cabling 303. Electrode 302 may be, for example, less than 2 millimeters (mm) in diameter. Cabling 303 includes an inner tubing 304 and a plurality of cables 305 each including a wire and associated insulation. A wire 306 included in cables 305 electrically couples to electrode 302, as described herein. Signals sent between electrode 302 and a device (e.g., pulse generator 150) via wire 306 facilitate controlling electrically stimulation delivered by electrode 302 and/or recording measurements (e.g., voltage measurements) measured at electrode 302.

Electrode 302 has a radially inner surface 310 and a radially outer surface 312. In this embodiment, two slots 314 are formed in electrode 302, extending from radially inner surface 310 to radially outer surface 312. Slots 314 define a strip 320 therebetween. In this embodiment, strip 320 includes a substantially planar portion 322. Alternatively, strip 320 may have any shape and/or configuration that enables electrode assembly 300 to function as described herein.

To electrically coupled wire 306 to electrode 302, a weld 330 is formed between electrode 302 and wire 306 on strip 320. Notably, weld 330 is formed on radially outer surface 312 of strip 320. Specifically, as shown in FIG. 3, wire 306 is threaded through slots 314 such that wire 306 is above (i.e., radially outward of) strip 320 but below (i.e., radially inward of) the remainder of electrode 302. Welding wire 306 to radially outer surface 312 provides several advantages. For example, once formed, weld 330 is readily visible for inspection purposes. Further, weld 330 is easier to form on radially outer surface 312 than radially inner surface 310.

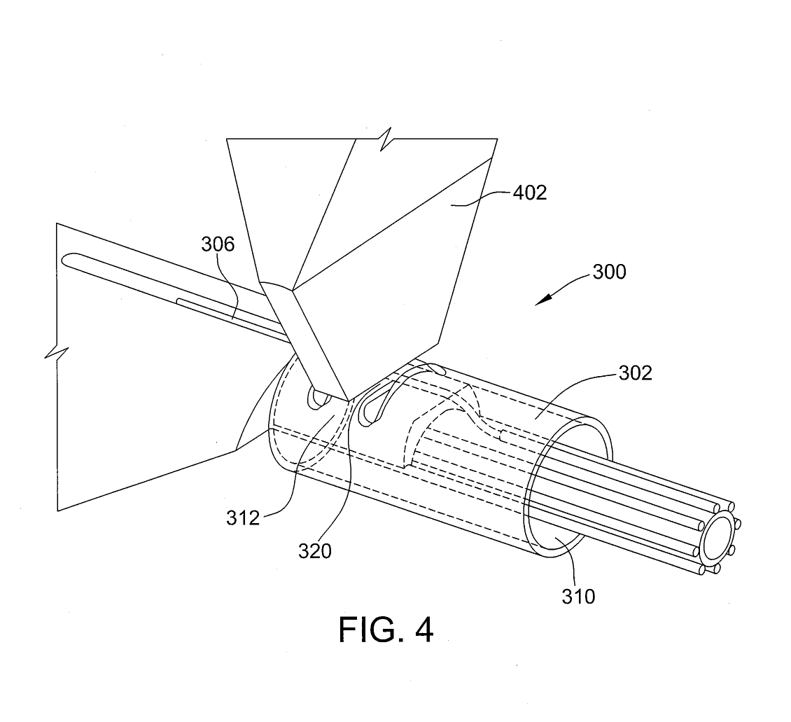

For example, FIG. 4 illustrates forming weld 330 using a resistance weld tool 402. Alternatively, weld 330 may be formed using laser welding or any other suitable welding technique (e.g., arc welding, gas welding, electron beam welding, or solid-state welding). As shown in 4, as wire 306 is welded to radially outer surface 312, the location of weld 330 is readily accessible to resistance weld tool 402. In contrast, if wire 306 were welded to radially inner surface 310, it would be relatively difficult, if not impossible, to position resistance weld tool 402 properly for the welding. Once weld 330 is formed, to secure wire 306, at least a portion of electrode assembly 300 is back-filled with a polymer using a reflow or injection molding process.

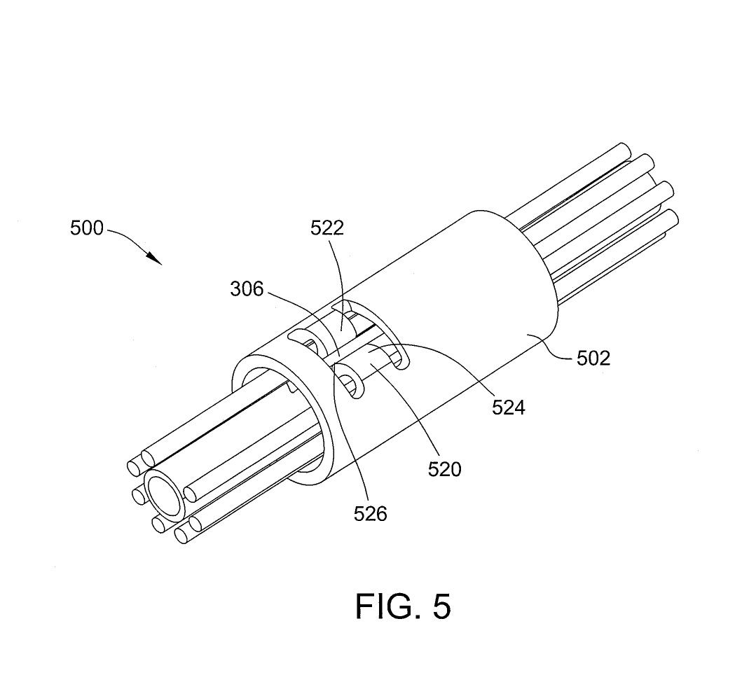

FIG. 5 is a perspective view of an alternative electrode assembly 500. Unless otherwise indicated, electrode assembly 500 is substantially similar to electrode assembly 300. In contrast to strip 320 of electrode assembly 300, a strip 520 of electrode 502 of assembly 500 does not include a substantially planar portion. Instead, strip 520 includes a first curved portion 522 and a second curved portion 524 that bend towards each other to meet at a midpoint 526 of strip 520. In this embodiment, wire 306 is welded to strip 520 proximate midpoint 526.

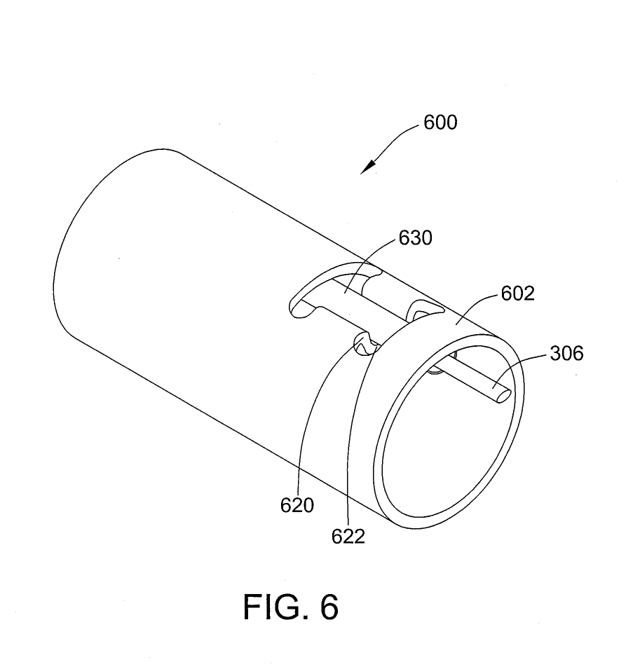

FIG. 6 is a perspective view of another alternative electrode assembly 600. Unless otherwise indicated, electrode assembly 600 is substantially similar to electrode assembly 300. In contrast to electrode assembly 300, in electrode assembly 600, wire 306 is not welded directly to a strip 620 of an electrode 602. Instead a conductive tubing 630 is crimped onto wire 306, and the combined conductive tubing 630 and wire 306 are welded onto electrode 602. In this embodiment, wire 306 still includes insulation. However, the heat from the weld destroys/flows the cable insulation to create the electrical connection. In other embodiments, the bare wire (i.e., without insulation) may be welded directly onto electrode 602. In the embodiment shown in FIG. 6, a planar portion 622 of strip 620 is radially recessed relative to the rest of electrode 602.

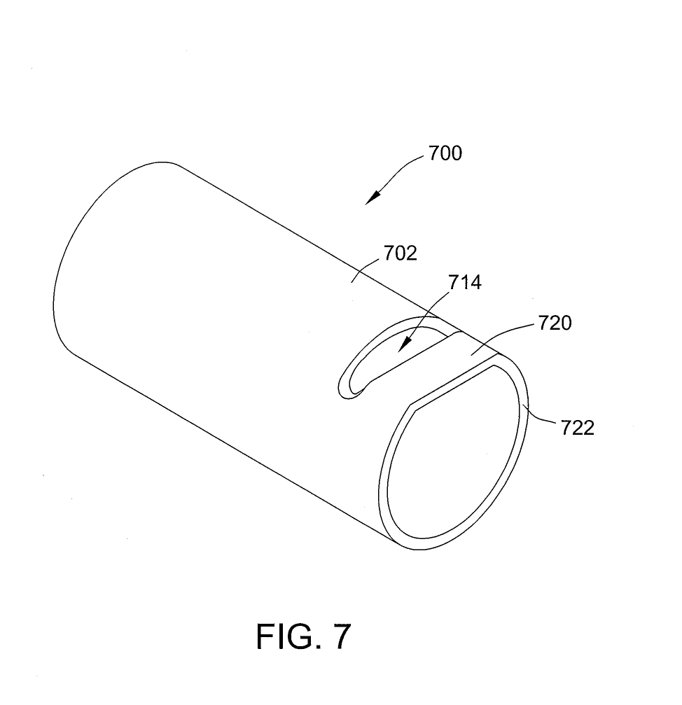

FIG. 7 is a perspective view of yet another alternative electrode assembly 700. Unless otherwise indicated, electrode assembly 700 is substantially similar to electrode assembly 300. In contrast to electrode assembly 300, a strip 720 is located at an end 722 of an electrode 702 such that electrode 702 includes only a single slot 714. Slot 714 may have a width of, for example, two thousandths of an inch. In the configuration of electrode assembly 700, it may be easier to position wire 306, as wire 306 need only be threaded through one slot 714, instead of multiple slots 314. Further, strip 720 may provide more surface area than in embodiments including multiple slots.

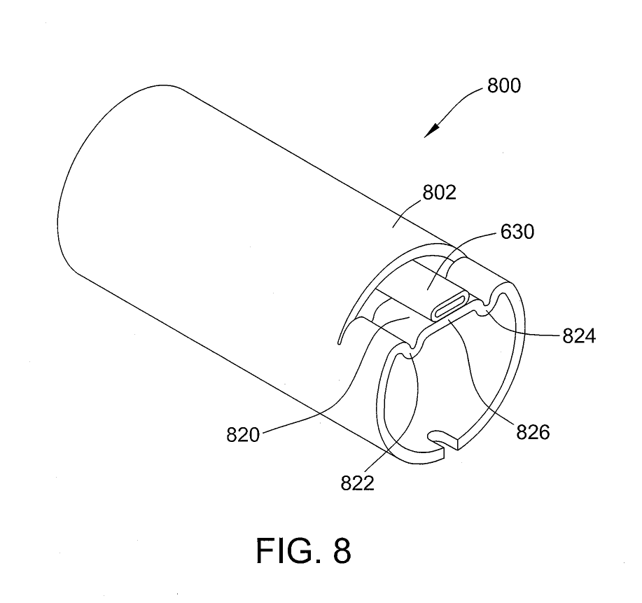

FIG. 8 is a perspective view of another alternative electrode assembly 800. Unless otherwise indicated, electrode assembly 800 is substantially similar to electrode assembly 700. Conductive tubing 630 is shown in FIG. 8 (and may also be used with electrode assembly 700) on a strip 820 of an electrode 802. In contrast to strip 720, strip 820 includes a first crimped feature 822, a second crimped feature 824, and a substantially planar portion 826 extending between first and second crimped features 822 and 824. Crimped features 822 and 824 facilitate improving a structural integrity of strip 820.

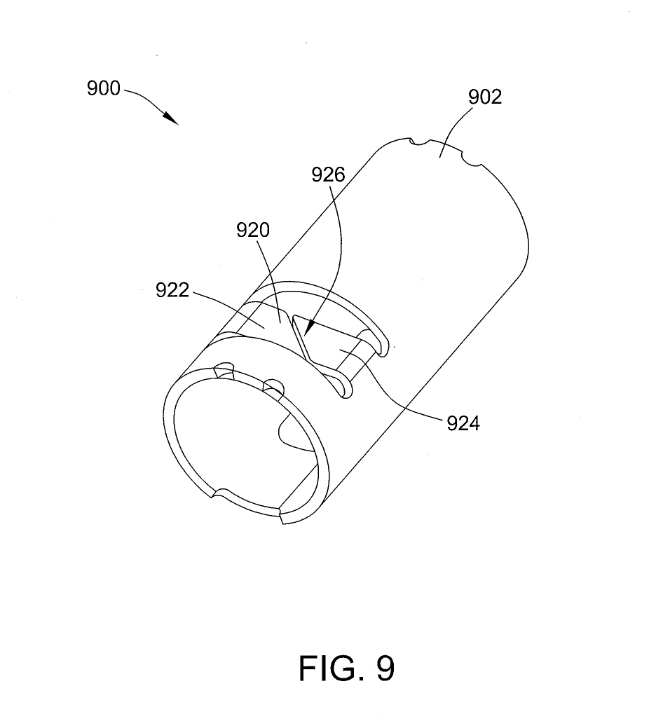

The electrodes described herein (e.g., electrodes 302, 502, 602, 702, and 802) may be fabricated using any suitable methods. For examples, the electrodes may be fabricated using a progressive die or a deep drawing technique. FIG. 9 is a perspective view of an electrode 902 formed using a progressive die. As shown in FIG. 9, for electrode 902, a strip 920 is formed by a first segment 922 and a second segment 924 extending towards one another and separated by a slit 926.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. An electrode assembly comprising:
a wire; and
a substantially cylindrical electrode comprising:
a radially inner surface;
a radially outer surface; and
a strip defined by at least one slot extending from the radially inner surface to the radially outer surface, wherein the wire is welded to the radially outer surface of the strip.

2. The electrode assembly of claim 1, wherein the strip comprises a substantially planar portion, and wherein the wire is welded to the substantially planar portion.

3. The electrode assembly of claim 1, wherein the strip is defined by a single slot.

4. The electrode assembly of claim 1, wherein the strip is defined by a first slot and a second slot.

5. The electrode assembly of claim 1, wherein the strip comprises:
a first crimped feature;
a second crimped feature; and
a substantially planar portion extending between the first and second crimped features.

6. The electrode assembly of claim 1, wherein the wire is welded directly to the strip, and wherein the wire comprises one of a bare wire and a wire including insulation.

7. The electrode assembly of claim 1, further comprising a conductive tubing crimped onto the wire, wherein the conductive tubing and the wire are welded to the strip.

8. A neurostimulation system comprising:
an implantable pulse generator;
a substantially cylindrical electrode comprising:
a radially inner surface;
a radially outer surface; and
a strip defined by at least one slot extending from the radially inner surface to the radially outer surface; and
a wire electrically coupling the implantable pulse generator to the electrode, wherein the wire is welded to the radially outer surface of the strip.

9. The neurostimulation system of claim 8, wherein the strip comprises a substantially planar portion, and wherein the wire is welded to the substantially planar portion.

10. The neurostimulation system of claim 8, wherein the strip is defined by a single slot.

11. The neurostimulation system of claim 8, wherein the strip is defined by a first slot and a second slot.

12. The neurostimulation system of claim 8, wherein the strip comprises:
a first crimped feature;
a second crimped feature; and
a substantially planar portion extending between the first and second crimped features.

13. The neurostimulation system of claim 8, wherein the wire is welded directly to the strip.

14. The neurostimulation system of claim 8, further comprising a conductive tubing crimped onto the wire, wherein the conductive tubing and the wire are welded to the strip.

15. A method of assembling an electrode assembly, the method comprising:
threading a wire through at least one slot defined in a substantially cylindrical electrode that includes a radially inner surface, a radially outer surface, and a strip defined by the at least one slot extending from the radially inner surface to the radially outer surface; and
welding the wire to the radially outer surface of the strip.

16. The method of claim 15, wherein welding the wire comprises welding the wire to a substantially planar portion of the strip.

17. The method of claim 15, wherein threading the wire comprises threading the wire through a single slot.

18. The method of claim 15, wherein threading the wire comprises threading the wire through a first slot and a second slot.

19. The method of claim 15, further comprising crimping a conductive tubing onto the wire, wherein welding the wire comprises welding the wire and the conductive tubing to the strip.

20. The method of claim 15, wherein welding the wire comprises welding the wire using one of resistance welding, laser welding, arc welding, gas welding, electron beam welding, and solid-state welding.
